(19) United States
(12) Patent Application Publication
(10) Pub. No.: US 2004/0131600 A1
(43) Pub. Date: Jul. 8, 2004

(54) Method of preventing adhesions by apoptosis of adhesion peritoneal cells

(76) Inventors: Michael P. Diamond, Grosse Pointe, MI (US); Ghassan M Saed, West Bloomfield, MI (US)

Correspondence Address: ROPES & GRAY LLP, ONE INTERNATIONAL PLACE, BOSTON, MA, 02110-2624, US

(21) Appl. No.: 10/471,899

(22) PCT Filed: Mar. 11, 2002
(86) PCT No.: PCT/US02/07119

(51) Int. Cl. A61K 48/00; C12Q 1/68

(52) U.S. Cl. 424/93.21; 514/44; 435/6

(57) ABSTRACT

Methods for the prevention of adhesion formation and development involve the administration of therapeutic formulations to a patient which modulates the rate of apoptosis of adhesion fibroblast cells. The formulations preferably include Bax, Bax enhancers, such as p53, Bax agonists, Bcl-2 inhibitors and Bcl-2 antagonists. A method is also provided for determining the predisposition of a subject to adhesion formation by measuring the Bcl-2/Bax ratio at multiple sites within the subject.

### BACKGROUND OF THE INVENTION

It is well established that injuries to the peritoneal surface of the peritoneum result in the development of post-operative adhesions in the vast majority of patients following surgery. The peritoneum is composed of mesothelial cells with submesothelial tissue containing fibroblasts, macrophages, endothelial cells, muscle cells, monocytes and blood vessels.

Cellular processes resulting in either normal peritoneal tissue repair or the development of adhesions include migration, proliferation and/or differentiation of several cell types, among them inflammatory, immune, mesothelial and fibroblast cells. Molecules produced locally by these cells regulate fibrinolytic activity, tissue remodeling and angiogenesis, as well as the synthesis and deposition of extracellular matrix material (ECM), and these processes are central to the development of adhesions.

The molecular events underlying peritoneal wound healing and the development of fibrous adhesions are complex, multifactoral and not well defined. The cascade of events that leads to peritoneal wound repair in many aspects resembles the events that occur during skin wound healing, which is characterized by inflammation, cellular migration, proliferation, phenotypic differentiation and tissue remodeling. During wound healing, fibroblasts invade the wound in the first few days of healing, and these fibroblasts have multiple functions important to wound repair. These functions include collagen synthesis, ECM reorganization, and wound contraction, resulting in mature scar formation.

Tissue remodeling involves the deposition and degradation of the extracellular matrix, which is a highly regulated process occurring during wound repair, and which is influenced by a host of locally expressed growth factors, cytokines and eicosanoids. The extracellular matrix is a dynamic component capable of modulating various cellular activities including cell-cell interaction, proliferation, differentiation and sequestration of potent biological response modifiers from the wound environment. In addition, excess production and deposition of the extracellular matrix is a key factor in producing tissue fibrosis throughout the body, including the development of peritoneal adhesions.

Apoptosis, or programmed cell death, is a component of normal development and differentiation in most tissues, including tissues involved in adhesion development. Apoptosis is a complex process occurring in a wide variety of organisms that removes aging or injured cells from the body. This type of cell death may be inhibited by deleterious stimuli, such as hypoxia, distorting the balance of cellular proliferation, differentiation and death, thereby impairing the normal peritoneal wound repair process. Indeed, a lower degree of apoptosis is found in dermal fibroblasts isolated from the skin of keloids and hypertrophic scars, as compared to normal skin fibroblasts.

There are at least two signaling pathways which are known to trigger apoptosis. The first is mediated by the interaction of membrane receptors and ligands, such as Fas ligand and TNF-α. The second pathway is triggered by exogenous stimuli such as hypoxia, radiation and chemotherapeutic drugs, wherein the death signal is transmitted through the mitochondria. This second pathway involves the altered expression of p53 and members of the Bcl-2 family. p53 point mutations have been detected in fibroblasts isolated from keloids, and the proapoptotic protein Bax which is expressed primarily in involved skin fibroblasts. In contrast to Bax, the antiapoptotic protein Bcl-2 has been found to be expressed at higher levels in fibroblast cells.

Hypoxia has a variety of effects on fibroblasts, both in vivo and in vitro. Hypoxia stimulates matrix synthesis with increased expression of fibronectin, and type I and type III collagens. Hypoxia also stimulates the production of a variety of growth factors, including the profibrotic factor transforming growth factor-β1 (TGF-β1) in human mesothelial and fibroblast cells. Although fibroblasts are invariably exposed to hypoxia in ischemic conditions, the effect of hypoxia on the apoptosis of human peritoneal and fibroblasts is not known.

The overexpression of TGF-β1 has been implicated in fibrotic disorders at various sites throughout the body, such as pulmonary fibrosis, glomerulonephritis, cirrhosis of the liver, and dermal scarring. Elevated levels of TGF-β expression occurs in adhesion tissues, in the peritoneal fluid of patients with adhesions, and in surgically induced adhesion formation in animal models. Mice that are heterozygous for TGF-β1 (+/−) have been shown to have significantly lower adhesions, and express at least two fold lower TGF-β1 protein in their peritoneal fluids, as compared with wild type (+/+) animals as early as 2 hours post-injury. See Krause et al., J. Invest Surg., 12, pages 31-38 (1999). Additionally, the postoperative peritoneal administration of TGF- β1 has been shown to increase the incidence of adhesion formation, while neutralizing antibodies directed against TGF-β reduce such incidence. Lucas et al., J. Surg. Res., 65, pages 135-138 (1996); and Williams et al., J Surg. Res., 52, pages 65-70 (1991).

It has been suggested that peritoneal adhesions develop in the vast majority of subjects, with more frequent occurrence in certain subjects following surgical procedures as opposed to others. The molecular basis for such predisposition is not known. Accordingly, it is an objective of this invention to provide a method for preventing or reducing the incidence of post-operative surgical adhesions with or without the use of barrier materials by addressing the molecular basis of the condition.

### SUMMARY OF THE INVENTION

It has now been discovered that the apoptosis rate is significantly higher in human normal peritoneal fibroblasts than in adhesion fibroblasts, and that hypoxia inhibits apoptosis and enhances the proliferation of adhesion fibroblasts in vivo. The decrease in the apoptotic process is accompanied by an increase in the Bcl-2/Bax expression ratio. Accordingly, the adhesion fibroblast phenotype appears to be more resistant to hypoxia-induced apoptosis than the normal peritoneal fibroblast phenotype, and this could be a defense mechanism by which this cell layer maintains critical functions in the presence of extreme environmental stress. These discoveries have led to the development of novel methods for treating surgical adhesions based on the molecular causes of adhesions.

In one aspect, the invention comprises a method for the prevention or remediation of surgical adhesions by treating a patient at risk of developing such adhesions with a therapeutic formulation to modulate the apoptosis of fibroblast cells in a subject predisposed to form adhesions. The formulations of this invention can be applied topically to the site of potential adhesion formation, or systemically. For example, the active ingredient in the formulations may be selected from the group consisting of Bax, Bax enhancers, such as p53, Bax agonists, Bcl-2 inhibitors and Bcl-2 antagonists.

In another embodiment, a method is provided for determining whether a human subject is predisposed to develop adhesions during or following surgery. This method comprises measuring the Bcl-2/Bax ratio in cells and tissue samples in a subject, comparing this ratio to Bcl-2/Bax ratios for normal peritoneal fibroblast cells and adhesion fibroblast cells, and determining whether the Bcl-2/Bax ratio of the subject, or at a site within the subject, such as an organ (e.g. ovary, uterus or bowel), or a tissue (e.g. abdominal wall or tendon sheath) is within a normal range. In accordance with this method, an increase in the Bcl-2/Bax ratio indicates a predisposition to develop adhesions.

In a further embodiment, this invention relates, broadly, to method for preventing or remediating the development or reformation of surgical adhesions by modulating the apoptosis in peritoneal cells in a subject. By “peritoneal cells” is meant, in the context of this invention, cells of the mesothelial lining of the peritoneum, including fibroblast cells, macrophage cells, endothelial cells, muscle cells, and monocytes.

An “enhancer,” as used herein, is a biological or chemical agent which increases the expression levels of a cytokine or protein in cells, such as peritoneal fibroblast cells.

### BEST MODES FOR CARRYING OUT THE INVENTION

### DETAILED DESCRIPTION OF THE INVENTION

Peritoneal mesothelial cells that line the serosal surface of the peritoneal cavity provide a natural protective barrier that prevents the organs from adhering to adjacent opposing surfaces. However, cellular or tissue injury that is induced following a surgical procedure, an infection or inflammation compromises the integrity of the mesothelial cells, and can result in a local biological response with the objective of repairing the defected surface. If a cellular or tissue injury is relatively extensive, it can lead to excess migration and proliferation of various wound cells, such as fibroblasts. This response initiates a cascade of events that often result in the development of peritoneal adhesions, known to be the major cause of bowel obstruction, pain and infertility, and hospital readmissions.

Hypoxia triggers apoptosis in fibroblast cell systems. The mechanism by which hypoxia induces apoptosis is believed to involve mitochondrial pathways, as opposed to ligand-receptor stimuli mediated by cytokines such as TNF-α or Fas ligand. In the former pathway, stimulation of cell death signals occurs through modulation of the expression of specific genes such as p53 and members of the Bcl-2 family. The Bcl-2 family of proteins plays a major role in the regulation of the apoptotic processes. Heterodimers and homodimers of the proapoptotic Bax protein, and the antiapoptotic Bcl-2 protein, determine cell survival or death by affecting the permeability of the mitochondrial membrane. The p53 protein upregulates the expression of Bax, and may also downregulate Bcl-2 protein expression, in tissues during cell apoptosis. The increase in Bcl-2 expression in adhesion fibroblast cells in response to hypoxia coincides with the decrease in Bax expression.

Adhesion reformation is known to occur more frequently than de novo adhesion formation. Tissue remodeling during the wound healing process is governed by the dynamic equilibrium between growth and programmed cell death, or apoptosis. The control of growth and apoptosis are intimately associated, and a disturbance of the balance between these two processes often leads to pathological events, such as cell accumulations in cancer cells and tissue fibrosis.

It has now been found that fibroblasts are exposed to hypoxia during ischemic events, and that the hypoxia inhibits apoptosis and enhances proliferation of peritoneum and adhesion, and adhesion fibroblast cells and tissue. By removing, or reversing, the effect of these inhibitory factors, the rate of apoptosis of adhesion fibroblasts can be increased, and adhesion formation can be impeded. This can be accomplished by, for instance, increasing the level of the Bax protein in the affected cells. Accordingly, Bax can be used as a therapeutic agent to reduce or prevent postoperative adhesions and tissue fibrosis. Alternatively, or additionally, suitable therapeutic formulations can include proteins which upregulate Bax expression in cells, such as p53. Further, proteins which downregulate Bcl-2 expression can also be used in the therapeutic formulations of this invention.

The administration of the preparations of the invention to potentially affected tissue and organs, locally or systemically, can induce protection against postoperative surgical adhesion development, or adhesion reformation. The preparations of the invention are useful for treating or preventing adhesions that form at a site and that have potential or actual deleterious effects. The preparations of the invention include Bax, Bax enhancers, Bax agonists and stimulatory agents, such as p53, and Bcl-2 inhibitors and antagonists.

Adhesions that can be successfully treated according to the method of this invention include, but are not limited to, primary and secondary adhesions in the following sites: in the abdominal cavity, including intestine to intestine, and intestine to peritoneum; in the pelvic cavity, including adhesion of the uterus, ovaries or fallopian tubes to other structures including each other and the pelvic wall; in tendons and their support structures, including tendon to synovium; in the repair of nerve sheaths; in the repair of the spinal column or disks; in the pericardium; in the treatment of joints for inflammation, and to prevent pannus formation; in the extraoccular muscle, to prevent adhesions from limiting the field of vision; and in any situation in which adhesions form that can impair function or cause pain.

The prevention of postoperative surgical adhesion development in a subject includes prophylactic treatment to prevent adhesion development following planned or elective surgical procedures, as well as following emergency operations. In addition to the surgical procedures described above, elective surgeries within the scope of this invention include the following intraabdominal surgeries: right hemicolectomy; left hemicolectomy; ovarian cystectomy, sigmoid colectomy; subtotal colectomy; total colectomy; laparoscopic or open cholecystectomy; hysterectomy, oophorectomy, salpingectomy, treatment of endometriosis, treatment of ectopic pregnancy, gastrectomy; pancreatectomy; splenectomy; liver, pancreas, small bowel, or kidney transplantation; lysis of adhesions; cesarean sections and other pelvic procedures, uterine surgery, etc. Emergency intraabdominal surgeries include those surgeries used to correct the following conditions: perforated ulcer (duodenal or gastric); perforated diverticulitis; obstructive diverticulitis; bowel obstruction; perforated appendicitis; blunt abdominal trauma; eye surgeries; penetrating abdominal trauma; ruptured abdominal aortic aneurysm, cardiac surgeries, open and endoscopic orthopedic surgeries, neurosurgeries, gynecologic and pelvic surgeries, and surgeries to correct wound infections.

The preparations of the invention are administered to a subject in an effective amount for inducing protection against postoperative surgical adhesion development. An “effective amount” for inducing protection against postoperative surgical adhesion development, as used herein, is that amount of pharmaceutical composition that will, alone or together with further doses or additional therapeutic compounds, inhibit or prevent the development of postoperative surgical adhesions.

The term “subject,” as used herein, means a human or non-human mammal, including but not limited to, a dog, cat, horse, cow, pig, sheep, goat, chicken, primate, rat, and mouse.

The terms “prevent” and “preventing” as used herein refer to completely or partially inhibiting a biological response, as well as inhibiting an increase in a biological response. For instance, the prevention of adhesion development refers to partially or completely inhibiting adhesion formation and adhesion reformation, as well as inhibiting an increase in adhesion formation and adhesion reformation. The preparations of the invention when administered “in conjunction with” a surgical procedure, are administered close enough in time with the surgery or trauma that predispose the host to adhesion development, so that a protective effect against the particular disorder is obtained. The preparations may be administered long before the surgery, e.g., in the case of elective surgery (i.e., weeks or even months), preferably with booster administrations closer in time to (and even after) the surgery. Particularly in emergency situations, the preparations may be administered immediately before (minutes to hours), during and/or after the surgery. It is important only that the preparation be administered close enough in time so as to enhance the subject's response against adhesions, thereby increasing the chances of a successful host response and reducing the likelihood of adhesion development.

The present invention provides pharmaceutical compositions for medical use, which in some aspects comprises the preparations of the invention together with one or more pharmaceutically acceptable carriers and optionally other therapeutic ingredients. Thus the invention may also include pharmaceutical compositions in combination with an anti-infectious agent such as an antibacterial or anti-viral agent, an anti-inflammatory agent, an antibiotic, or other therapeutic agent, and a pharmaceutically acceptable carrier. The pharmaceutical compositions useful in the invention may be delivered separately with the other therapeutic agents, or in the form of therapeutic cocktails. A therapeutic cocktail includes a mixture of the pharmaceutical composition of the invention and another therapeutic agent. In this embodiment, a common administration vehicle (e.g., tablet, implant, injectable solution, etc.) contains both the pharmaceutical composition and another therapeutic agent. Alternatively, the other therapeutic agent can be separately dosed if desired. A barrier material, such as hyaluronic acid or carboxymethyl cellulose, can also be used as a carrier for the compositions of this invention.

The precise amount of the therapeutic agent used in combination with the pharmaceutical compositions of the invention depends upon a variety of factors, including the particular pharmaceutical composition selected, the dose and dose-timing selected, the mode of administration, the nature of any surgical or medical procedure contemplated, and the characteristics of the subject. Where local administration is carried out, it will be understood that very small amounts may be required (nanograms and possibly picograms). The precise amounts selected can be determined without undue experimentation, particularly since a threshold amount is any amount which will favorably enhance the response.

Multiple doses of the pharmaceutical compositions of the invention are contemplated. For instance, when being administered in conjunction with a surgical procedure, the compositions of the invention can be administered in multiple doses over a three week to one day period preceding surgery. Further, doses may be administered post surgery as well. Any regimen that prevents or retards the development of adhesions may be used, although optimum doses and dosing regimens are those that would not only inhibit the development of adhesion formation, but also would result in complete protection against adhesion development. Desired time intervals for the delivery of multiple doses of a particular pharmaceutical composition can be determined by one of ordinary skill in the art employing no more than routine experimentation.

The formulations of the invention are administered in pharmaceutically acceptable solutions, which may routinely contain pharmaceutically acceptable concentrations of salt, buffering agents, preservatives, compatible carriers, adjuvants, and optionally other therapeutic ingredients.

Suitable buffering agents include: acetic acid or its salt (1-2% w/v); citric acid or its salt (1-3% w/v); boric acid or its salt (0.5-2.5% w/v); succinic acid; and phosphoric acid or its salt (0.8-2% w/v). Suitable preservatives include benzalkonium chloride (0.003-0.03% w/v); chlorobutanol (0.3-0.9% w/v); parabens (0.01-0.25% w/v) and thimerosal (0.004-0.02% w/v).

The pharmaceutical compositions of the invention contain an effective amount of a pharmaceutical composition optionally included in a pharmaceutically acceptable carrier. The term “pharmaceutically acceptable carrier” means one or more compatible solid or liquid filler, dilutants or encapsulating substances which are suitable for administration to a human or other animal. The term “carrier” denotes an organic or inorganic ingredient, natural or synthetic, with which the active ingredient is combined to facilitate the application of the composition to the subject. The components of the pharmaceutical compositions also are capable of being commingled with the pharmaceutical compositions of the present invention, and with each other, in a manner such that there is no interaction which would substantially impair the desired pharmaceutical efficiency.

Compositions suitable for parenteral administration conveniently comprise sterile aqueous preparations, which can be isotonic with the blood of the recipient. Among the acceptable vehicles and solvents are water, Ringer's solution, and isotonic sodium chloride solution. In addition, sterile, fixed oils are conventionally employed as a solvent or suspending medium. For this purpose any bland fixed oil may be employed including synthetic mono- or di-glycerides. In addition, fatty acids such as oleic acid, find use in the preparation of injectables. Carrier formulations suitable for subcutaneous, intramuscular, intraperitoneal or intravenous administrations may be found in Remington's Pharmaceutical Sciences, Mack Publishing Company, Easton, Pa.

The pharmaceutical compositions useful in the invention may be delivered in mixtures of more than one pharmaceutical composition. A mixture may consist of several pharmaceutical compositions.

A variety of administration routes are available. The particular mode selected will depend, of course, upon the particular pharmaceutical composition, the particular condition being treated, and the dosage required for therapeutic efficacy. The methods of this invention, generally speaking, may be practiced using any mode of administration that is medically acceptable, meaning any mode that produces effective levels of an immune response without causing clinically unacceptable adverse effects. Preferred modes of administration include parenteral, injection, infusion, deposition, implantation, anal or vaginal supposition, oral ingestion, inhalation, topical administration. Injections can be intravenous, intradermal, subcutaneous, intramuscular, or interperitoneal. For example, the pharmaceutical composition can be injected directly into the surgical site for the prevention of adhesions. In some embodiments, the injections can be given at multiple locations. Implantation includes inserting implantable drug delivery systems, e.g., microspheres, hydrogels, polymeric reservoirs, cholesterol matrixes, polymeric systems, e.g., matrix erosion and/or diffusion systems and non-polymeric systems, e.g., compressed, fused, or partially-fused pellets. Inhalation includes administering the pharmaceutical composition with an aerosol in an inhaler, either alone or attached to a carrier that can be absorbed. For systemic administration, it may be preferred that the pharmaceutical composition is encapsulated in liposomes. The term “parenteral” includes subcutaneous injections, intravenous, intramuscular, intraperitoneal, intrasternal injection or infusion techniques.

In certain preferred embodiments of the invention, the administration can be designed to result in the sequential exposure of the pharmaceutical composition over some period of time, e.g., hours, days, weeks, months or years. This can be accomplished by repeated administrations of the pharmaceutical composition, by one of the methods described above, or alternatively, by a sustained-release delivery system in which the pharmaceutical composition is delivered to the subject for a prolonged period without repeated administrations. By sustained-release delivery system, it is meant that the total release of the pharmaceutical composition does not occur immediately upon administration, but rather is delayed for some period of time. Release can occur in bursts, or it can occur gradually and continuously. Administration of such a system can be, e.g., by long-lasting oral dosage forms, bolus injections, transdermal patches, and subcutaneous implants.

Examples of systems in which release occurs in bursts includes, e.g., systems in which the pharmaceutical composition is entrapped in liposomes which are encapsulated in a polymer matrix, the liposomes being sensitive to specific stimuli, e.g., temperature, pH, light or a degrading enzyme, and systems in which the pharmaceutical composition is encapsulated by an ionically-coated microcapsule with a microcapsule core degrading enzyme. Examples of systems in which release of the pharmaceutical composition is gradual and continuous include, e.g., erosional systems in which the pharmaceutical composition is contained in a form within a matrix, and effusional systems in which the pharmaceutical composition permeates at a controlled rate, e.g., through a polymer. Such sustained release systems can be e.g., in the form of pellets, or capsules.

In one particular embodiment, the preferred sustained release device is a biocompatible microparticle or microencapsulated product or implant that is suitable for implantation or administration to the mammalian recipient. Exemplary bioerodible implants that are useful in accordance with this method are described in PCT International application no. PCT/US/03307 (Publication No. WO 95/24929, entitled “Polymeric Gene Delivery System”). The polymeric matrix preferably is in the form of a microparticle, such as a microsphere (wherein the pharmaceutical composition is dispersed throughout a solid polymeric matrix), or a microcapsule (wherein the pharmaceutical composition is stored in the core of a polymeric shell). Other forms of the polymeric matrix for containing the pharmaceutical composition include films, coatings, gels, implants, and stents. The size and composition of the polymeric matrix device is selected to result in favorable release kinetics in the tissue into which the matrix is introduced. The size of the polymeric matrix further is selected according to the method of delivery which is to be used, typically injection into a tissue. The polymeric matrix composition can be selected to have both favorable degradation rates and also to be formed from a material which is bioadhesive, to further increase the effectiveness of transfer when the matrix is administered to a mucosal surface. The matrix composition also can be selected not to degrade, but rather to release by diffusion over an extended period of time. The biocompatible microsphere may be suitable for oral delivery. Such microspheres are disclosed in Chickering et al., Biotech. And Bioeng., (1996) 52:96-101; Mathiowitz et al., Nature, (1997) 386:.410-414; and PCT Patent Application WO 30 97/03702.

Both non-biodegradable and biodegradable polymeric matrices can be used to deliver the pharmaceutical compositions to the subject. Biodegradable matrices are preferred. Such polymers may be natural or synthetic polymers. The polymer is selected based on the period of time over which release is desired, generally in the order of a few hours to a year or longer. Typically, release over a period ranging from between a few hours and three to twelve months is most desirable. The polymer optionally is in the form of a hydrogel that can absorb up to about 90% of its weight in water, and further optionally is cross-linked with multi-valent ions or other polymers.

Bioadhesive polymers of particular interest include bioerodible hydrogels described by H. S. Sawhney, C. P. Pathak and J. A. Hubell in Macromolecules, (1993) 26:581-587, the teachings of which are incorporated herein; casein, gelatin, glutin, polyanhydrides, polyacrylic acid, alginate, chitosan, poly(methyl methacrylates), poly(ethyl methacrylates), poly(butylmethacrylate), polyhyaluronic acids, poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), and poly(octadecyl acrylate).

Other sustained release delivery systems useful according to the invention include, but are not limited to, fatty acids and a medicinal pump. Preferably the fatty acids are C₉-C₂₀ fatty acids.

The compositions may conveniently be presented in unit dosage form and may be prepared by any of the methods well known in the art of pharmacy. Such methods include the step of bringing the pharmaceutical composition into association with a carrier which constitutes one or more accessory ingredients. In general, the compositions are prepared by uniformly and intimately bringing the pharmaceutical composition into association with a liquid carrier, a finely divided solid carrier, or both, and then, if necessary, shaping the product. The pharmaceutical composition may be stored in a lyophilized condition.

The pharmaceutical compositions can be suspended in a liquid, e.g., in dissolved form or colloidal form. The liquid can be a solvent, partial solvent, or non-solvent. In many cases, water or an organic liquid can be used. The pharmaceutical compositions are administered to the subject in a therapeutically-effective amount. By therapeutically-effective amount it is meant that amount which is capable of at least partially preventing, reversing, reducing, decreasing, ameliorating, or otherwise suppressing adhesions. A therapeutically-effective amount can be determined on an individual basis and is based, at least in part, on considerations of the age, sex, size, and health of the subject; the type of pharmaceutical composition used, the type of delivery system used; the time of administration; and whether a single, multiple, or controlled-release dose regimen is employed. A therapeutically-effective amount can be determined by one of ordinary skill in the art employing such factors and using no more than routine experimentation.

The dosage concentration of the pharmaceutical composition actually administered is dependent, at least in part, upon the final concentration of pharmaceutical composition that is desired at the site of action, the method of administration, the efficacy of the particular pharmaceutical composition, the longevity of the particular pharmaceutical composition, and the timing of administration. Preferably, the dosage form is such that it does not substantially deleteriously affect the subject. The dosage can be determined by one of ordinary skill in the art employing such factors and using no more than routine experimentation.

The following examples serve to illustrate the invention without limiting it thereby. It will be understood that variations and modifications can be made without departing from the spirit and scope of the invention.

### EXAMPLE 1

Harvested tissue samples are placed in standard media (DMEM medium containing 10% fetal bovine serum, 2% penicillin and streptomycin). Tissues are cut into small pieces in sterile culture dish and transferred into another fresh T-25 flask with 3 ml of dispase solution (2.4 U/ml, GIBCO BRL, Life Technologies, Inc.). The flasks are incubated overnight at 37° C. in an environ-shaker (LAB-LINE Instruments, Inc.). Samples are then centrifuged for 5 min. at 1400 g, transferred into a fresh T-25 flask with pre-warmed DMEM medium, and put in 37° C. incubator (95% air and 5% CO₂). The outgrowth of fibroblasts generally takes 2 weeks. When confluence is reached, the cells are transferred to 90-mm tissue culture dishes and cultured in standard media with 10% FBS. Thereafter, the confluent dishes are subcultured by trypsinization (1:3 split ratio) to a aximal of 12th passage. Studies are conducted using passage 3-5 cells to maintain comparability.

### Hypoxic Culture Conditions

All hypoxic experiments are performed in an airtight Plexiglas chamber (Bellco Glass, Vineland, N.J.). The chamber is deoxygenated by positive infusion of 2% CO₂/nitrogen gas mixture. Cultures are then placed in a standard humidified tissue incubator. There is no statistically significant difference in viability by crystal violet or trypsan blue exclusion. Parallel cultures are placed in nomoxia for all time points. Cells are harvested at 24 hours time point. All experiments are performed in triplicate.

### In Situ Analysis of DNA Integrity (Tunel Assay)

Peritoneal and adhesion fibroblasts are grown in Lab-Tek chamber slides (Nunc Inc., Naperville, Ill.). The Tunel technique is performed as described by the Promega Apoptosis Detection System. Slides are treated with proteinase K (20 μg/ml) in 10 mM tris HCI (pH 8.0) for 15 min at room temperature and washed 4 times for 2 min in ddH₂O. Endogenous peroxidase is inactivated by incubating the sections for 5 min in 3% H₂O₂ at room temperature, and then washing 3 times in ddH₂O. Slides are preincubated for 10 min at room temperature in TdT buffer (30 mM Tris-HCl [pH 7.2]-140 mM Na cacodylate-1 mM cobalt chloride), and incubated in a moisture chamber for 1 hr. at 37° C. with 20-30 μl of TdT buffer with 0.5 U TdT/μl and 40 μM fluorescein 12-dUTP. The reaction is stopped by transferring the slides to 2×SSC buffer (300 mM NaCl-30 mM sodium citrate) for 15 min at room temperature followed by the addition of propidium iodide to stain all cells. Detection of localized green fluorescence of apoptotic cells (fluorescein 12-dUTP) and in red background (propidium iodide) is performed by fluorescence microscopy. Quantitative analysis is performed by FACS of the changes in mean fluorescence for cell suspensions. Positive controls are performed by treating cells with DNase I (Iμg/ml) in TdT buffer for 10 min at room temperature before incubation with biotinylated nucleotide.

### Determination of Bcl-2/Bax mRNA Ratio by Multiplex RT/PCR

The multiplex RT/PCR technique is utilized to compare mRNA levels of Bcl-2 and Bax molecules in peritoneal and adhesion fibroblasts in response to hypoxia treatment. The advantage of the multiplex RT/PCR technique is that products are normalized to a housekeeping gene in the same tube. This normalization confers a number of benefits including standardization of i) reverse transcription and PCR efficiencies, ii) pipetting differences between reactions, and iii) differences in template input.

RT/PCR: RNA is isolated using the monophasic solution of phenol and GITC/Trizol method.

Removal of DNA contamination from RNA: RNA is treated with Dnase (RNase-free) in 10 mM Tris-C 1, pH8.3, 50 mM KCL, 1.5 mM MgCl₂ in the presence of RNase ribonuclease inhibitor, and incubated at 37° C. for 30 min. After extraction with phenol/chloroform and ethanol precipitation, the RNA is redissolved in DEPC-treated water.

cDNA synthesis: 20 μg of total RNA is heated to 68° C. for 10 min in the presence of 2 μl oligo dT primer, and then rapidly chilled on ice. A master mix containing 4 μl 5X first strand buffer, 2 μl 0.1M dithiothreitol, 1 μl 10 mM dNTP mix, 1 μl superscript II (200 U; Life Technologies, Inc.), and 1 μl RNase inhibitor is added and each reaction is incubated at 42° C. for 1 hour.

PCR Amplification: Aliquots from the cDNA reaction is PCR-amplified in 100 μl reaction as follows: 10 μl of 10X PCR reaction buffer, 1 mM from each of the deoxynucleoside triphosphate, 20 μM from each primers and 2.5 U Taq polymerise enzyme. Mineral oil is added to prevent evaporation. The reaction is initiated by heat denaturation at 95° C. for 1 min, annealing the primers for 2 min at 60° C. (depends on the primer combination), and then extension for 3 min at 72° C. This is repeated for 35 cycles using PCR (Perkin-Elmer). After the final cycle, the temperature is maintained at 72° C. for 7 min to allow completion of synthesis of amplified products. Analysis of PCR-amplified products is by fractionation over a 2% agarose gel followed by ethidium bromide staining of DNA bands. Scanning densometer is used to determine the ratio of intensity of each band relative to β-actin. Densometric analysis of gel bands is performed using NTH image analysis program.

Primer design and controls: Optimal oligonucleotide primer pairs for multiplex RT/PCR amplification of oligo dT-primed reverse-transcribed cDNA are selected with the aid of the computer program Oligo 4.0 (National Biosciences, Inc., Plymouth, Minn.). The following human oligonucleotide primers, which amplify variable portions of the protein coding regions, are used:

| | | | |
|---|---|---|---|
| Locus | Sense (5′-′3) | Antisense (5′-′3) | bp |

| | | | |
|---|---|---|---|
| B- | AAGCAGGAGTATGACGAGT | GCCTTCATACATCTCAAGT | 559 |
| actin | CCG | TGG | |
| Bcl-2 | TGTGGTATGAAGCCAGACC | CAGGATAGCAGCACAGGAT | 153 |
| | TCC | TGG | |
| Bax | TTCTGACGGCAACTTCAAC | AGGAAGTCCAATGTCCAGCC | 135 |
| | TGG | | |

Since quantitative application of this method is contingent upon the analysis of the PCR products during amplification phase, prior to the plateau, cycle relationships and dilutional curves for cDNA for each molecule and the housekeeping gene β-actin are determined empirically.

### Statistical Analysis

A one-way ANOVA with Student-Newman-Keuls post-hoc comparisons are conducted using SPSS V.10.0 for windows.

The effect of varying the time of hypoxia on apoptosis of peritoneal and adhesion fibroblasts (n=3) is determined. With the use of the Tunel assay, hypoxia for 24 h is found to cause a marked increase in peritoneal fibroblast DNA fragmentation, compared with cells exposed to standard conditions. However, 24 h hypoxia causes a marked decrease in DNA fragmentation of adhesion fibroblasts, compared with cells exposed to standard conditions. Longer exposure to hypoxia up to 72 h does not further reduce the level of apoptosis. Cultures of up to 24 h in hypoxia are therefore used to compare with standard conditions, and complete detailed quantification of the apoptosis that occurs in the first 24 h in culture. With the use of the Flow Cytometry technique to quantitate apotosis, apoptosis is higher in fibroblasts of normal peritoneum (mean florescent channel=18.32±1.1) than adhesions (mean florescent channel=14.11±0.94) of the same patient in response to hypoxia treatment (P<0.001).

The Bcl-2 and Bax protein family have been implicated in the mechanism of hypoxia-induced apoptosis in a variety of tissues. The multiplex RT/PCR technique has been developed to simultaneously amplify Bcl-2, Bax and β-actin mRNAs. Multiplex RT/PCR is of great practical benefit by allowing normalization of the quantity of specific mRNA target to levels of a constitutively expressed target such as the housekeeping gene β-actin. Multiplex RT/PCR is performed as described herein. The intensity of each band is normalized to β-actin band using a scanning densitometer. The results show that adhesion fibroblasts have almost double the Bcl-2/Bax mRNA ratio as compared to peritoneal fibroblasts. In particular, hypoxia resulted in a 25% decrease in the Bcl-2/Bax ratio for normal peritoneum fibroblasts, indicating an increase in apoptosis. In contrast, hypoxia resulted in a 33% increase in the Bcl-2/Bax ratio for adhesion fibroblasts, indicating a decrease in apoptosis. The Bcl-2/Bax ratio can determine whether cells will die by apoptosis, or be protected from it. The higher the Bcl-2/Bax mRNA ratio, the lower the apoptosis rate.

The Flow Cytometry technique is also utilized to measure the proliferative fraction, which measures proliferation of cells, in normal peritoneal and adhesion fibroblasts before and after exposure to 24 h hypoxia. Adhesion fibroblasts show a 2-fold increase in their proliferative fraction as compared to peritoneal fibroblasts. Hypoxia treatment results in a further increased up to 3.5-fold in the proliferative fraction of adhesion as compared to peritoneal fibroblasts.

### EXAMPLE 2

The purpose of this example is to determine whether tissue hypoxia, which results from tissue injury during surgery, lowers the rate of apoptosis of peritoneal fibroblasts during peritoneal healing. These fibroblasts remain in the proliferative stage and do not die through apoptosis. Therefore, these cells continue to produce extracellular molecules (ECM), resulting in adhesion development.

Fibroblast primary cultures are obtained from normal peritoneal and adhesion tissues of the same patient. The fibroblasts are cultured under normal peritoneal and adhesion conditions for 24 hours before evaluating apoptosis by the Tunel assay. The Tunel assay measures the fragmented DNA of apoptotic cells by incorporating fluoroscein-12-dUTP at 3′-OH DNA ends using the enzyme Terminal Deoxynucleotidyl Transferase (TdT). The fluoroscein-labeled DNA is visualized directly by fluorescence microscopy, and quantified by flow cytometry (FACS).

Hypoxia results in an increase in the rate of apoptosis in peritoneal fibroblasts, but a decrease in the rate of apoptosis of adhesion fibroblasts. FACS analysis shows that apoptosis is higher in fibroblasts of normal peritoneum (MFC=18.32) than adhesions (MFC=14.11) of the same patient in response to hypoxia treatment.

### EXAMPLE 3

The purpose of this example is to identify the expression pattern of molecules involved in the regulation of apoptosis in human normal peritoneal and adhesion fibroblasts.

Human fibroblast cultures were prepared as described in Example 1. A genearray was prepared using a human apoptosis GEArray kit (Supper Ary, Inc.) to characterize the gene expression profiles associated with fibroblasts isolated from normal peritoneal and adhesion tissues. The kit was composed of 23 apoptosis-related genes and two housekeeping genes, actin and GADPH. The total RNA was isolated using the monophasic solution of phenol and the GITC/Trizol method as previously described. RNA was converted to cDNA in the presence of chemiluminescent dATP. The labeled cDNA was hybridized to the membrane that contained the 23 apoptosis-related genes, and then exposed to an x-ray film to visualize the expressed genes.

Table 1 below shows the apoptosis-related genes that are differentially expressed in normal and adhesion fibroblasts. The intensity of each band was assigned a score. A score of (+5) has the highest expression. A score of (−) indicates no expression. As shown in Table 1, adhesion fibroblasts are characterized by the upregulation of the antiapoptotic proteins p53, Mdm-2 and Bcl-x; and the down regulation of the proapoptotic proteins Gadd-45 and Caspase-3.

TABLE 1

| | Normal | Adhesion |
|---|---|---|
| Apoptosis Markers | Peritoneal Fibroblasts | Fibroblasts |
| p53 | — | +5 |
| Gadd-45 | +4 | +1 |
| Mdm-2 | +2 | +4 |
| Bcl-x | +2 | +4 |
| Caspase-3 | +3 | +1 |

### EXAMPLE 4

The purpose of this example is to test the hypothesis that fibroblasts isolated from adhesion tissues have abnormal apoptosis regulation by comparing the expression of the p53 gene in fibroblasts isolated from normal peritoneal and adhesion tissues.

Human fibroblast cultures were prepared as described in Example 1. Peritoneal cells were fixed in 100% cold acetone for 10 minutes, and then allowed to air dry for 30 minutes at room temperature. The slides were then placed on modified PBS for 10 minutes. P53 (DO7, Vector Laboratories, Burlingame, Calif.) diluted 1:10 was placed on the slides for 2 hours at room temperature. Slides were rinsed in PBS and incubated with biotinylated secondary antibody and avidin-biotin complex for 10 minutes each. After color development with 3-amino-9-ethylcarboaole, the slides were counterstained with hematoxylin.

The p53 protein was found to be absent in normal peritoneal fibroblasts, but present in significantly higher levels in adhesion fibroblasts .

Each of the foregoing patents, patent applications and references that are recited in this application are herein incorporated in their entirety by reference. Having described the presently preferred embodiments, and in accordance with the present invention, it is believed that other modifications, variations and changes will be suggested to those skilled in the art in view of the teachings set forth herein. It is, therefore, to be understood that all such variations, modifications, and changes are believed to fall within the scope of the present invention as defined by the appended claims.

1. A method for the prevention or remediation of surgical adhesions comprises modulating the apoptosis in peritoneal cells in a subject.

2. The method of claim 1 wherein the peritoneal cells are fibroblast cells.

3. The method of claim 1 wherein the cells are adhesion cells which contribute to adhesion development.

4. The method of claim 1 which further comprises treating a patient at risk of developing such adhesions with a therapeutic formulation which increases the rate of apoptosis.

5. The method of claim 4 wherein the therapeutic formulation contains an active ingredient selected from the group consisting of Bax, Bax enhancers, Bax agonists, Bcl-2 inhibitors and Bcl-2 antagonists.

6. The method of claim 5 wherein the Bax enhancer is p53.

7. The method of claim 1 wherein the therapeutic formulation is locally administered to the site of potential adhesion formation or adhesion reformation.

8. The method of claim 1 wherein the method comprises a gene therapy method which introduces the p53 or Bax gene into the fibroblast cells.

9. A method of determining whether a human subject, or an organ or tissue site within a subject, is predisposed to develop adhesions during or following surgery comprising measuring the Bcl-2/Bax ratio in cells and tissue samples in a subject, comparing this ratio to Bcl-2/Bax ratios for normal peritoneal fibroblast cells and adhesion fibroblast cells, and determining whether the Bcl-2/Bax ratio of the subject is within a normal range.

10. The method of claim 9 wherein an increase in the Bcl-2/Bax ratio indicates a predisposition to develop adhesions.
